(19) United States
(12) Patent Application Publication
(10) Pub. No.: US 2020/0406014 A1
(43) Pub. Date: Dec. 31, 2020

(54) EAR CATHETER

(71) Applicant: Holger Sudhoff, Bielefeld (DE)

(72) Inventor: Holger Sudhoff, Bielefeld (DE)

(21) Appl. No.: 16/968,493

(22) PCT Filed: Feb. 7, 2019
(86) PCT No.: PCT/EP2019/052987

§ 371 Date: Aug. 7, 2020

(30) Foreign Application Priority Data
Feb. 9, 2018 (DE) 10 2018 102 937.3

(51) Int. Cl. A61M 31/00; A61M 25/10; A61M 25/06; A61M 39/10

(52) U.S. Cl. CPC A61M 31/00; A61M 25/10; A61M 2025/1052; A61M 39/10; A61M 2210/0668; A61M 25/0662

(57) ABSTRACT

The invention relates to a non-invasive ear catheter (1) for introduction into the eustachian tube, comprising a catheter tube (2), an expandable balloon (3) for the occlusion of the eustachian tube, and an injection channel (5) for introducing medication into the middle ear region.

The invention relates to an ear catheter for the non-invasive introduction into the eustachian tube, which enables medications to be delivered to the middle ear region over a longer period of time.

Numerous diseases of the middle and inner ear are known that are to be treated with medications. In the event a topical treatment is required, this can be performed through the eardrum on the one hand and through the eustachian tube on the other. Treatment of the inner ear can also be carried out via the middle ear region. However, the administration of medication in liquid form into the eustachian tube or middle ear usually has not a long-lasting effect, due to the fact that the medication drains through the eustachian tube into the nasopharyngeal space.

A series of infections, inflammations but also the treatment of an acute hearing loss or Meniere's disease requires long-term treatment, which may last for days or may even take a few weeks. As a rule, new medication, such as a corticosteroid, is constantly added in this case. This treatment is both time-consuming and requires regular and permanent medical supervision and care.

For this reason, it would be desirable to have available an apparatus by means of which a drug can be applied into the middle ear where it can be maintained for a longer period of time. Such an application should to the extent possible be non-invasive, that is should not be detrimental or damaging to the eardrum. In this case, only natural access via the nasopharynx and the eustachian tube must be resorted to.

This objective is achieved with the aid of an ear catheter of the kind first mentioned above, which comprises a catheter tube, an expandable balloon for closing off toe eustachian tube and an injection channel for introducing medication Into toe middle ear region.

Such an ear catheter can be inserted non-invasively through the nose and the nasopharyngeal space into the eustachian lube and placed there in such a way that the balloon seals the eustachian tube after expansion. This occlusion interrupts the connection between the middle ear area located distal to the balloon and the nasopharynx. In this context, distal denotes the part of the ear catheter pointing towards the auditory system and the middle ear, while proximal refers to the part pointing away from the auditory system and the middle ear.

The ear catheter proposed by the present the invention is a balloon catheter, which in principle is built like a balloon catheter used for vasodilatation; however, said catheter does not serve to dilate the eustachian tube, but to occlude it. In this respect, the balloon of the ear catheter is only subjected to a minimal overpressure in such a way that a cuff is formed which seals off the eustachian tube (low-pressure cuff). For this purpose, a single balloon is sufficient. The tube of the ear catheter has a length of approximately 15 cm which is sufficiently long for the catheter to be inserted through one nostril and the nasopharyngeal space into the eustachian tube, with the proximal tube end remaining in the patients cheek area.

The injection channel extends essentially inside the ear catheter, except for its proximal end, which protrudes laterally from the ear catheter. The ear catheter and the injection channel are fitted with connectors for customary syringes, which allow filling the ear catheter with pressure medium and introducing medication solution Into the Inner ear. Expediently, said connectors are Luer lock style connections as they are commonly used for medical purposes, to which various syringes can be hooked up using appropriate equipment.

Customary materials as they are commonly employed for balloon catheter technological purposes can be used for the balloon, catheter and injection channel. These are plastic materials approved for medical applications, for example PVC, polyethylene, polypropylene and the like. The materials should be sufficiently elastic/flexible to allow insertion into the eustachian tube.

Advantageously, the catheter should be equipped with a handling aid for control. It is, moreover, advantageous to provide a curved distal end on the catheter, which enables the control and positioning of the balloon and catheter end into and within the eustachian tube to be facilitated. Such a curved end is called “pig tail” in catheters and guidewires and is widely used.

It goes without saying that the ear catheter is provided with a sealing device retaining the applied pressure medium after filling thus making sure the expanded state of the system is maintained. Similarly, the injection channel has a closure device at either its distal or proximal end that prevents the injected medication from draining out of the injection channel. Making use of the inflated balloon that occludes the eustachian tube, the injection channel which may consist of a soft plastic tube can be forcibly pressed together and thus sealed off.

The ear catheter the invention proposes is inserted into the eustachian tube in a non-invasive manner through the nose and the nasopharyngeal space. The insertion can be monitored by ear microscopy. As soon as the expandable balloon has reached the part of the eustachian tube to be occluded, a liquid is applied to the balloon in the usual way causing it to expand. It is considered advisable to design the balloon as a low-pressure cuff and to provide a pressure limiter by means of which the pressure is expediently kept at a maximum of 1 bar (gauge pressure). For the expansion of the balloon a liquid is used, preferably a physiological saline solution. For the introduction of the saline solution, the proximal connection of toe ear catheter is fitted with a conventional Luer lock to which a syringe for the saline solution can be attached. To enable toe balloon to be kept pressurized, the catheter tube is equipped with a one-way valve.

The injection channel, usually an injection tube, extends through the catheter tube and toe balloon and readies into toe middle ear region. At its proximal end it also equipped with a Luer lock connection for a syringe through which the medication can be injected Into the middle ear region. At toe distal end there is an outlet, which can be atraumatically designed and fitted with a one-way valve. With a one-way valve, toe outlet opens under toe pressure of toe injected solution, if the tube is made of a soft, elastic material, even toe pressure exerted by toe balloon may be sufficient to seal it off. In this case, the balloon pressure causes toe injection tube to be forcibly pressed together and sealed and the tube only opens as a result of the pressure exerted by toe injection fluid when it is inserted with the syringe. As an alternative to arranging a one-way valve distally it may as well be located proximally.

As provided by toe invention, the ear catheter is particularly suitable for administering antibiotic solutions to the middle ear area, which is advisable in toe treatment of persistent bacterial infections. The standard treatment with corticosteroids to attend an acute loss of hearing can also be appropriately carried out with such an ear catheter. The standard therapy approach requires that such a corticosteroid be administered over a longer period of time, usually for about two weeks, with daily re-application being necessary as the drug drains away into toe nasopharyngeal space. Here, the usual treatment period amounts to 14 days. Using the ear catheter proposed by toe invention a much more intensive and shorter treatment can be realized, as the catheter makes sure toe medication is constantly present where it is needed. This shortens the duration of treatment considerably, possibly to a few days. In particular, the catheter suitably causes the medication to be retained in the middle ear region for a longer period of time, especially even for several days.

In addition, antiinflammatory drugs may also be introduced via the ear catheter.

The ear catheter is placed in position through one of the nasal openings and, after applying pressure and infusing the medication, is fixed to the cheek with a suitable patch. It remains in the eustachian lube during the treatment. The patient remains mobile, a longer stay in a clinic is not necessary, regular medical supervision is sufficient Ear microscopy can be used for supervision.

When the treatment has been completed, the ear catheter can be easily removed after pressure has been relieved.

The ear catheter also allows the treatment of diseases of the inner ear, because the drugs can usually pass through the membrane of the round window and ingress the inner ear region. This is due to a diffusion process.

The invention is explained in more detail by way of the enclosed FIGURE.

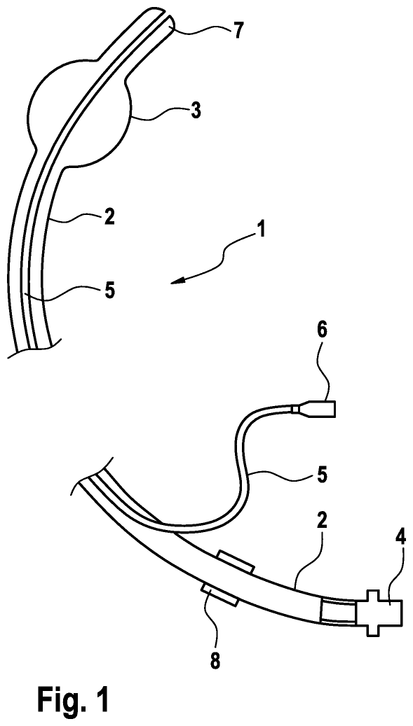

The inventive ear catheter 1 according to FIG. 1 consists of a catheter tube 2, which ends distally in an expandable balloon 3 (cuff). On the proximal end, there is a Luer lock connection 4 for a conventional syringe, by means of which physiological saline solution can be introduced with a view to expanding balloon 3. An interposed pressure gauge (not shown) serves to monitor the pressure and limits it to 1 bar.

An injection tube 5 extends through the center of the catheter, said tube also being provided with a luer lock connection 6, via which a medication solution can be injected using a syringe. The injection channel 5 terminates in an atraumatically designed outlet opening or tip 7, which opens under the pressure of the injection fluid and closes automatically after injection has been completed.

The ear catheter t has a total length of about 15 cm; the diameter of the catheter tube 2 amounts to 3 mm and the injection tube diameter is about 1 mm.

A sleeve 8, which is frictionally mounted on the catheter, serves as a maneuvering and handling aid. The sleeve is preferably made of plastic material and is appropriately slotted so that it can be fitted or removed.

In the treatment of a case of acute hearing loss, after the catheter has been appropriately positioned, and by using, for example, a 2 ml syringe, a cortisone solution is filled into the middle ear and left there for the required time. Riling of the middle ear Is monitored by ear microscopy. The procedure is preferably performed under local anesthesia, but in special cases a general anesthetic may be advisable. After the medication has been administered, the proximal part of the application catheter is stuck to the cheek with a patch. The pressure is released from the injection channel. The catheter balloon remains in place in the eustachian tube and prevents the Injected solution from draining away. At the end of the treatment, the pressure is released from the balloon and the catheter is withdrawn.

1. Non-invasive ear catheter for introduction into the eustachian tube, comprising a catheter tube (2), an expandable balloon (3) for the occlusion of the eustachian tube, and an injection channel (5) for introducing medication into the middle ear region.

2. Ear catheter according to claim 1, characterized in that the injection channel (5) extends inside the catheter.

3. Ear catheter according to claim 1, characterized in that the expandable balloon (3) is designed as a low-pressure cuff.

4. Ear catheter according to claim 1, characterized by a pressure limiter for a maximum pressure of 1 bar.

5. Ear catheter according to claim 1, characterized in that the injection channel (5) is provided with a Luer lock connection (6) for a syringe containing injection material.

6. Ear catheter according to claim 1, characterized in that the catheter tube (2) is provided with a Luer lock connection (4) for a liquid medium for the expansion of the balloon (3).

7. Ear catheter according to claim 6, characterized in that the liquid for expanding the balloon (3) is a physiological NaCl solution.

8. Ear catheter according to claim 1, characterized in that the injection channel (5) ends distally from the balloon (3).

9. Ear catheter according to claim 1, characterized in that the injection channel (5) is provided with an outlet opening (6) that is of atraumatic design.

10. Ear catheter according to claim 9, characterized in that the outlet opening (6) is of self-closing design.

11. Ear catheter according to claim 1, characterized by a handling aid (8).

12. Ear catheter according to claim 11, characterized in that the handling aid (8) is a plastic sleeve frictionally mounted on the catheter tube.

13. Ear catheter according to claim 1 to be employed for the non-invasive application of medication into the middle ear of a patient.

14. Ear catheter according to claim 13, characterized in that it is suitable for retaining the medication in the middle ear for several days.
